(19) United States
(12) Patent Application Publication
(10) Pub. No.: US 2012/0308673 A1
(43) Pub. Date: Dec. 6, 2012

(54) COMPOSITION COMPRISING THE EXTRACT OF PRUNELLA VULGARIS L. FOR PREVENTING AND TREATING ADHD DISEASE AND THE USE THEREOF

(76) Inventors: Jong Hoon Ryu, Seoul (KR); Jae Hoon Cheong, Namyangju-si (KR); Chan Young Shin, Seoul (KR); Se Jin Park, Geumsan-gun (KR)

(21) Appl. No.: 13/514,363

(22) PCT Filed: Nov. 10, 2010
(86) PCT No.: PCT/KR10/07904

§ 371 Date: Jun. 7, 2012

(30) Foreign Application Priority Data
Dec. 23, 2009 (KR) 10-2009-0129811

(51) Int. Cl. A61K 36/536; A61P 25/00

(52) U.S. Cl. 424/745

(57) ABSTRACT

The present invention relates to a composition comprising the extract of Prunella vulgaris L for preventing and treating ADHD and the use thereof.

### BACKGROUND OF THE INVENTION

1. Technical Field

The present invention relates to a composition comprising the extract of Prunella vulgaris L for preventing and treating ADHD disease and the use thereof.

2. Background Art

The main syndromes of ADHD (Attention Deficit and Hyperactivity Disorder), a frequently occurring psychological disorder on children are inattention, hyperactivity and impulsivity. There have been reported that the syndromes are accompanied by various psycho-social disorders such as learning disorder, juvenile delinquent, drug abuse and crime etc (Y. B. Lee, J. S. Shin, Korean Journal of family Social Work, pp 129-157, 2000; G. D. Kewley, British Medical Journal, 23, pp 1594-1596, 1998; L. B. Silver, Attention-deficit hyperactivity disorder, Washinton DC.: American psychiatry Press, Inc., 1992).

The pathological etiology of ADHD has been postulated to be genetic factor (R. A. Barley, Attention-deficit hyperactivity disorder: A Handbook of Diagnosis and Treatment. New York: Guilford Press. 1990); a reaction against the lead level or food additive of Instant food; Washington DC (G David, J. Neal, Abnormal Psychology, John Wiley & Sons. 1976).: environmental factors such as the relation between parents and their children, social position of parents, or drug abuse and smoking during pregnancy however it has been not identified yet (H. Mang, The Journal of Elementary Education, 18, pp 243-Z77, 2005).

Recently, ADHD has been reported to be closely involved in the functional disorder in fronto-striatal tract in the structural and functional study on brain imaging study using by ADHD patients and the drug effect due to MPH (Methylphenidate) is correlated with the functional change of dopaminergic system at the region (M. H. Teicheher, C. M. Anderson et al., Nat. Med., 6, pp 470-473, 2000; J. B. Schweitzer, D. O. Lee et al., Neuropsychopharmacology 28, pp 967-973, 2003).

Additionally, the behavior of ADHD patients has been reported to give rise to the unbalance between the control of noradrenergic nerve system and dopaminergic nerve system and it has been caused by the decreased effect of the catecholamines governing glutamatergic and GABAnergic neurons on the post synapse (E. B. Johansen, P. R. Killeen et al., Behav. Brain Func. 3, pp 60, 2007).

Ritalin® and Methylphen® comprising methylphenidate have been used as frequently prescribed CNS stimultants and reported to be effective therapeutic method as a short-term therapy to alleviate the inattention, hyperactivity and impulsivity of ADHD patients. However, the methods for long-term therapy have not verified till now (S. Kim, D. Ahn, Y. Lee, J. Korean Neuropsychiatr. Assoc., 4, pp 683-699, 1998).

Accordingly, there has been needed to develop novel therapeutic agents showing long-term treating activity with little adverse action till now.

Prunus vulgaris L, Prunella vulgaris var. aleutica, Prunella vulgaris var. asiatica and Prunella vulgaris var. lilacina belonged to Labiatae have been used as a treating agent to treat hepatic cirrhosis, hypertension, fever in Asia and Europe (Psotov J. et al; Biological activities of Prunella vulgaris extract. Phytother Res. 17 (2003), pp. 1082-1087).

Recently, there have been reported that the several triterpene compounds isolated from the extract of Prunella vulgaris showed anti-allergic activity and anti-inflammatory activity (Ryu, S. Y. et al., Anti-allergic and anti-inflammatory triterpenes from the herb Prunella vulgaris. Planta Medica, 66, (2000), pp 358-360) as well as anti-oxidative activity, anti-microbial activity and anti-viral activity (Europe (Psotov J. et al; Biological activities of Prunella vulgaris extract. Phytother Res. 17 (2003), pp. 1082-1087).

However, there has been not reported or disclosed on the therapeutic effect for ADHD of the extract of Prunus vulgaris, L. (Labiatae) in any of the above cited literatures, the disclosures of which are incorporated herein by reference.

To investigate an inhibitory effect of the extract of Prunus vulgaris, L. on the attention deficit, hyperactivity and impulsivity through in vivo tests, the inventors of the present invention have intensively carried out in vivo tests such as ADHD animal model test, for example, SHR (spontaneous hypertensive rat) model test, and spontaneous alternation behavior test using by Y-maze test etc, and finally completed present invention by confirming that the extract inhibited the attention deficit, hyperactivity and impulsivity of the tested rats.

These and other objects of the present invention will become apparent from the detailed disclosure of the present invention provided hereinafter.

### SUMMARY OF THE INVENTION

Accordingly, it is another object of the present invention to provide a pharmaceutical composition comprising the extract of Prunus vulgaris, L as an active ingredient in an amount effective to treat or prevent ADHD disease, together with a pharmaceutically acceptable carrier.

It is another object of the present invention to provide a use of the extract of Prunus vulgaris, L. in the manufacture of a medicament employed for treating or preventing ADHD disease in a mammal.

It is the other object of the present invention to provide a method of treating or preventing ADHD disease in a mammal wherein method comprises administering to said mammal an effective amount of above described extract, together with a pharmaceutically acceptable carrier thereof.

### DISCLOSURE OF THE INVENTION

In one embodiment of the present invention, the present invention provides a pharmaceutical composition comprising the extract of Prunus vulgaris, L as an active ingredient in an amount effective to treat or prevent ADHD disease, together with a pharmaceutically acceptable carrier.

Additionally, the present invention provides a use of the extract of Prunus vulgaris, L. in the manufacture of a medicament employed for treating or preventing ADHD disease in a mammal.

Additionally, the present invention provides a method of treating or preventing ADHD disease in a mammal wherein method comprises administering to said mammal an effective amount of the extract of Prunus vulgaris, L, together with a pharmaceutically acceptable carrier thereof.

Additionally, the present invention also provides a health care food comprising the extract of Prunus vulgaris, L, as an active ingredient in an amount effective to improve ADHD disease, together with a sitologically acceptable additives.

The term, “extract' defined herein comprise the extract prepared by extracting a spike, herb, flower, or root, preferably, spike of Prunus vulgaris, L, with polar solvent, for example, water, lower alcohol such as methanol, ethanol, butanol etc and the mixture thereof, preferably, water or the mixture solvent with ethanol and water, more preferably, water or 60-95% ethanol.

The pharmaceutical composition of the present invention can contain about 0.01˜50% by weight of the above extract based on the total weight of the 5 composition.

The health food of the present invention comprises above extracts as 0.01 to 80%, preferably 1 to 50% by weight based on the total weight of the composition.

The above-described health care food can be contained in health food, health beverage etc, and may be used as powder, granule, tablet, chewing tablet, capsule, beverage etc.

Hereinafter, the present invention is described in detail.

An inventive extract of Prunus vulgaris, L, can be prepared in detail by following procedures,

The inventive extract of Prunus vulgaris, L, can be prepared by follows; For example, the collected spike of Prunus vulgaris, L, is dried, cut, crushed and mixed with 1 to 100-fold, preferably, approximately 5 to 20-fold volume of distilled water, lower alcohols such as methanol, ethanol, butanol and the like, or the mixtures thereof, preferably, water or the mixture solvent with ethanol and water, more preferably, water or 60-95% ethanol; the solution is treated with hot water at the temperature ranging from 30 to 110° C., preferably from 50 to 100° C., for the period ranging from 1 hour to 24 hours, preferably, 1 hour to 5 hours with extraction method selected from the extraction method of hot water extraction, cold water extraction, reflux extraction, or ultra-sonication extraction, preferably, reflux extraction, or ultra-sonication extraction with 1 to 5 times, preferably 2 to 3 times, consecutively; the residue is filtered to obtain the supernatant to be concentrated with rotary evaporator, and then dried by vacuum freeze-drying, hot air-drying or spray drying to obtain dried extract powder of Prunus vulgaris L of the present invention.

Accordingly, in an another embodiment of the present invention, the present invention provides a method for preparing the inventive extract of Prunus vulgaris, L, comprising the step of; extracting the spike of Prunus vulgaris, L, with 1 to 100-fold volume of distilled water, lower alcohols such as methanol, ethanol, butanol and the like, or the mixtures thereof, at the temperature ranging from 30 to 110° C., for the period ranging from 1 hour to 5 hours, with the extraction method selected from reflux extraction or ultra-sonication extraction; filtering the residue to obtain the supernatant and concentrating and drying the supernatant to obtain dried extract powder of Prunus vulgaris L of the present invention.

It have been proved that the extract of Prunus vulgaris, L. prepared by the above-described method showed potent inhibition effect on the attention deficit, hyperactivity and impulsivity through in vivo tests, such as ADHD animal model test, for example, SHR (spontaneous hypertensive rat) model test and spontaneous alternation behavior test using by Y-maze test etc.

Accordingly, the extract of Prunus vulgaris, L. prepared by the above-described method can be useful in treating or preventing ADHD disease as well as in solving the various problems such as psycho-social disorders of children such as learning disorder and in preventing juvenile delinquent, drug abuse and crime etc.

Therefore, the present invention provides a pharmaceutical composition comprising the extract of Prunus vulgaris, L prepared by the above-described method as an active ingredient in an amount effective to treat or prevent ADHD disease, together with a pharmaceutically acceptable carrier.

Additionally, the present invention provides a use of the extract of Prunus vulgaris, L prepared by the above-described method in the manufacture of a medicament employed for treating or preventing ADHD disease in a mammal.

Additionally, the present invention provides a method of treating or preventing ADHD disease in a mammal wherein method comprises administering to said mammal an effective amount of the extract of Prunus vulgaris, L prepared by the above-described method, together with a pharmaceutically acceptable carrier thereof.

Additionally, the present invention also provides a health care food comprising the extract of Prunus vulgaris, L prepared by the above-described method, as an active ingredient in an amount effective to improve ADHD disease, together with a sitologically acceptable additive.

The term “prevent” defined herein means the inhibition of such those diseases in a mammal which is prone to be caught by those disease and the term “treat” used herein means (a) the inhibition of the development of disease or illness; (b) the alleviation of disease or illness; or (c) the elimination of disease or illness.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the present invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract of the present invention varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 0.1 to 5 g/kg by weight/day of the inventive extract or compounds of the present invention. The dose may be administered in single or divided into several times per day. In terms of composition, the complex herbal composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Accordingly, it is another object of the present invention to provide a health care food comprising the extract of Prunus vulgaris, L and a sitologically acceptable additive to prevent and alleviate ADHD disease.

Accordingly, it is the other object of the present invention to provide a health food or food additive comprising the extract of Prunus vulgaris, L to prevent and alleviate ADHD disease.

The term “a health care food” defined herein is “the food containing inventive extract of the present invention showing no specific intended effect but general intended effect in a small amount of quantity as a form of additive or in a whole amount of quantity as a form of capsule, pill, tablet etc”.

The term “a sitologically acceptable additive” defined herein is “any substance the intended use which results or may reasonably be expected to result-directly or indirectly-in its becoming a component or otherwise affecting the characteristics of any food” for example, thickening agent, maturing agent, bleaching agent, sequesterants, humectant, anticaking agent, clarifying agents, curing agent, emulsifier, stabilizer, thickner, bases and acid, foaming agents, nutrients, coloring agent, flavoring agent, sweetner, preservative agent, antioxidant, etc, which had been well-known in the art.

If a substance is added to a food for a specific purpose in that food, it is referred to as a direct additive and indirect food additives are those that become part of the food in trace amounts due to its packaging, storage or other handling.

The term “a health food” defined herein can be contained in various candy, health beverage, gum, tea, vitamin complex, or dietary food etc, and may be used as a form of powder, granule, tablet, chewing tablet, capsule, beverage etc for preventing or improving aimed disease.

Also, inventive extract can be added to food or beverage for prevention and improvement of aimed disease. The amount of inventive extract in food or beverage as a functional health food or health care food may generally range from about 0.01 to 15 w/w % of total weight of food for functional health food composition. In particular, although the preferable amount of inventive extract of the present invention in the functional health food, health care food or special nutrient food may be varied in accordance to the intended purpose of each food, it is preferably used in general to use as a additive in the amount of inventive extract of the present invention ranging from about 0.01 to 5% in food such as noodles and the like, from 40 to 100% in health care food on the ratio of 100% of the food composition.

To develop for health food, examples of addable food comprising above extracts of the present invention are various food, beverage, gum, vitamin complex, health improving food and the like, and can be used as power, granule, tablet, chewing tablet, capsule or beverage etc.

Also, the extract of the present invention will be able to prevent and improve ADHD disease by way of adding to child and infant food, such as modified milk powder, modified milk powder for growth period, modified food for growth period.

Above described composition therein can be added to food, additive or beverage, wherein, the amount of above described extract in food or beverage may generally range from about 0.1 to 80w/w %, preferably 1 to 50 w/w % of total weight of food for the health food composition and 1 to 30 g, preferably 3 to 10 g on the ratio of 100 ml of the health beverage composition.

Above described composition therein can be added to food, additive or beverage for prevention of aimed disease, For the purpose of preventing aimed disease, wherein, the amount of above described extract of the present invention in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described extract of the present invention as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 1000 of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Examples of addable food comprising aforementioned extract therein of the present invention are various food, beverage, gum, vitamin complex, health improving food and the like.

It will be apparent to those skilled in the art that various modifications and variations can be made in the compositions, use and preparations of the present invention without departing from the spirit or scope of the invention.

### BRIEF DESCRIPTION OF THE DRAWINGS

The above and other objects, features and other advantages of the present invention will more clearly understood from the following detailed description taken in conjunction with the accompanying drawing, in which;

FIG. 1 shows the inhibitory effect of an extract of Prunus vulgaris on the movement distance (hyperactivity) of SHR animal model;

FIG. 2 depicts the inhibitory effect of an extract of Prunus vulgaris on the movement duration (hyperactivity) of SHR animal model;

FIG. 3 presents the inhibitory effect of an extract of Prunus vulgaris on the alteration behavior of SHR animal model;

FIG. 4 represents the inhibitory effect of an extract of Prunus vulgaris on the impulsivity modulation of SHR animal model.

### BEST MODE FOR CARRYING OUT THE INVENTION

The following Example and Experimental Examples are intended to further illustrate the present invention without limiting its scope.

### EXAMPLE 1

### Preparation of an Extract of Prunus vulgaris L.

1-1. Preparation of Water Soluble Extract

1 kg of dried spike of Prunus vulgaris L purchased from Kyung-dong Market located in Seoul was cut into the pieces and pulverized with mixer. The powder was mixed with 10 L of distilled water and extracted with hot water for 2 hours at 100° C. using by heater (MS-E104, TOPS Co. Ltd.). The residue is filtered to obtain the supernatant and dried with freeze-dryer (FDU-2000, EYELA) to obtain 22.03 g of water soluble extract (designated as “PVW” hereinafter; yield-22.03%).

The sample was kept at −75° C. in refrigerator and used in following experiments by dissolving in distilled water before use.

1-2. Preparation of 70% Ethanol Soluble Extract

1 kg of dried spike of Prunus vulgaris L purchased from Kyung-dong Market located in Seoul was cut into the pieces and pulverized with mixer. The powder was mixed with 10 L of 70% ethanol and performed to ultra-sonication extraction for 2 hours at 60° C. using by ultrasonication apparatus (Powersonic 420, Hwashin Tech. Co. Ltd. www.hstech.co.kr). The residue is filtered to obtain the supernatant and dried with freeze-dryer (FDU-2000, EYELA) to obtain 16.85g of 70% ethanol soluble extract (designated as “PV80E” hereinafter; yield-16.85%).

The sample was kept at −75° C. in refrigerator and used in following experiments by dissolving in distilled water before use.

### REFERENCE EXAMPLE 1

### Preparation & Reagent

### 1-1. Animal

For following in vivo test, male ICR mice weighing 23-25 g purchased from Orient Co. Ltd. (Seoul, Korea) were acclimated into the environment for 5 days and freely accessible to water and forage in clean cage (Uimyung research institute for neuroscience in Sahmyook Uni. www.syu.ac.kr) and the cage was kept with following condition maintaining the temperature of 23±2° C. and the relative humidity of 55±10° C. under the regularly controlled light/dark condition with an interval of 12 hours.

### 1-2. Statistics

All test results were analyzed by one-way analysis of variance (ANOVA) followed by Student-Newman-Keults test for multiple comparisons and the statistical significance was set at P<0.05.

### EXPERIMENTAL EXAMPLE 1

### ADHD Activity Test (In Vivo)

### 1-1. Effect on Hyperactivity of SHR Animal Model

To test the inhibitory effect of the extract of Prunus vulgaris L prepared in Example 1 on the hyperactivity of SHR animals, following test was performed according to the procedure disclosed in the literature (Kong, W. X et al., Effect on taurine on rat behaviors in three anxiety models, (2006), Pharmacology Biochem., Behavior 83:271-276; Arulmozhi D. K. et al, Pharmacological studies of the aqueous extract of Sapindus trifoliatus on central nervous system: possible ant-migraine mechanism, J. Ethnophamacol., 97:491-496).

The rats were acclimated with the environment for 1 week and with apparatus more than once for 10 mins prior to test. PV70E and PVW prepared in Example 1 used as test groups as well as methylphenidate (Johnson Mattey Public Limited Co.) used as a positive control were intraperitoneally administered into mice in a dose of 3 mg/kg.

30 mins after the treatment with test samples, the SHR mice, i.e., ADHD animal models, and normal mice used as negative control were placed on the test box to examine the movement of mice and to determine the total movement distance and total movement duration of the mice for 10 mins using by analysis apparatus (EthoVision system; Noldus IT b.v., Netherland).

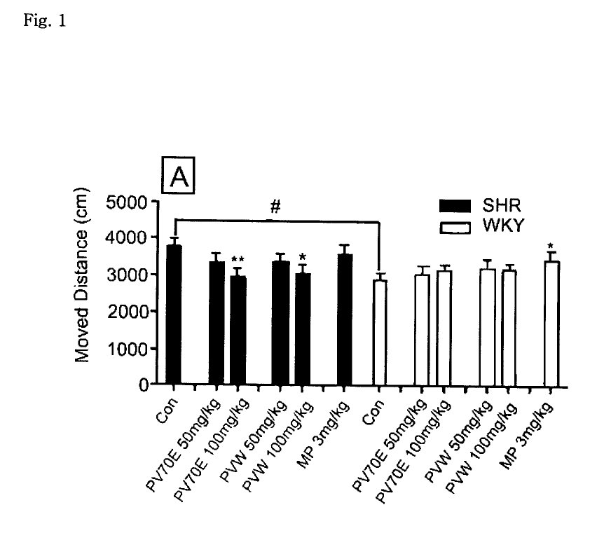

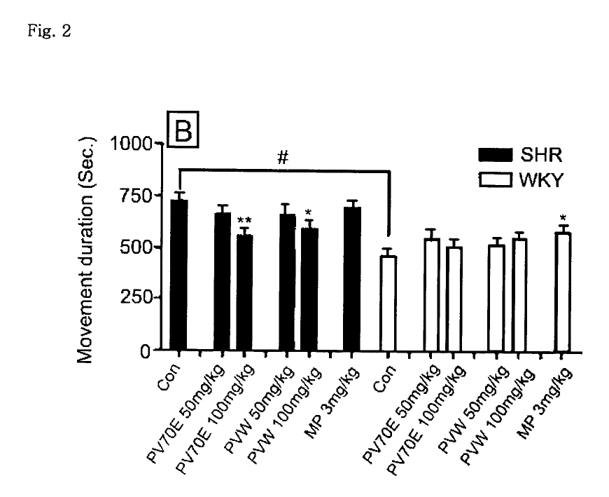

At the result, as can be seen in FIGS. 1 and 2, the general movement of SHR treated with the extract of Prunus vulgaris L prepared in Example 1 (PV70E and PVW) significantly decreased the increased movement of the SHR mice in respect to total movement distance and rearing number (significance; p<0.01, 0.05), of which movement of the SHR mice was increased comparing to normal mice (significance; p<0.01). However the extract of Prunus vulgaris L prepared in Example 1 (PV70E and PVW) did not affect any movement on normal mice.

In a while, methylphenidate (Johnson Mattey Public Limited Co.) used as a positive control increased the movement of normal mice whereas did not affect on the movement of SHR, which means that methylphenidate, a commonly used as a ADHD treating agents, may did not affect the hyperactivity or cause unfavorable effect on the hyperactivity of ADHD patients.

### 1-2. Effect on Alternation Behavior of SHR Animal Model

To test the inhibitory effect of the extract of Prunus vulgaris L prepared in Example 1 on the alternation behavior of SHR animals, following modified Y-maze test was performed according to the procedure disclosed in the literature (Sarter M., et al., Attenuation of scopolamine-induced impairment of spontaneous alternation by antagonist but not agonist and agonist beta-carbolines, (1988), Psychopharmacology (Berl). 94:491-495; Kim D. H. et al, Gomisin A improves scopolamine-induced memory impairment in mice, (2006), Eur. J. Pharmacol., 542:129-235).

The Y-maze test was conducted in a three-arm maze with angles of 120° between the arms designates as A, B and C respectively, which were 42 cm long and 3 cm wide with walls that were 12 cm high each. The maze floor and walls were constructed from dark opaque polyvinyl plastic. Mice were initially placed within one arm and the sequence and number of arm entries were recorded manually for each mouse over an 8 min period. The percentage of triads in which all the arms were represented, i.e., ABC, CAB or BCA but not BAB, was recorded as an ‘alternation’ to estimate short-term memory. Control group animals received 0.9% saline solution and scopolamine (1 mg/kg, i.p.) or vehicle was introduced to induce memory impairment 30 mins before the test. The arms were cleaned with water spray between tests to remove odors and residues. The alternation score (%) for each mouse was calculated according to following math formula 1:

alternation behavior (%)=(actual alternation)/(maximum alternation)×100 [Math formaule]

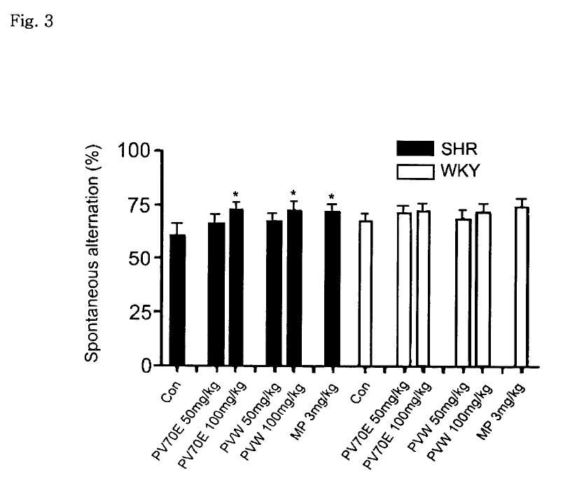

At the result, as can be seen in FIG. 3, the alternation behavior of SHR treated with the extract of Prunus vulgaris L prepared in Example 1 (PV70E and PVW) significantly increased the decrease alternation behavior of the SHR mice in a dose dependent manner (significance; p<0.05), of which alternation behavior of the SHR mice was decreased comparing to normal mice. The alternation behavior of normal mice (WKY white mice) did not significantly changed and the concentration capacity of SHR treated with the extract of Prunus vulgaris L prepared in Example 1 showed equivalent potency with that of SHR treated with methylphenidate used as a positive control.

### 1-3. Effect on Impulsivity Modulation of SHR Animal Model

To test the inhibitory effect of the extract of Prunus vulgaris L prepared in Example 1 on the impulsivity modulation of SHR animals, following modified test was performed according to the procedure disclosed in the literature (Bull, E., et al., Evaluation of the spontaneously hypertensive rat as a model of attention defici hyperactivity disorder: acquisition and performance of the DRL-60s test, (2000), Behavioural Brain Research 109:27-35; Marco, E. M. et al., Enhancement of endocannabinoid signaling during adolescence: Modulation of impulsivity and long-term consequences on metabolic brain parameters in early maternally deprived rats, (2007), Pharmacology Biochemistry and Behavior, 86:Issue 2, pp 334-345).

The test box consisting of three compartments, i.e., (a) a safety compartment, (b) an electronic current running compartment, (c) drink providing compartment and video-tracking system apparatus monitoring the test box were used in the experiment.

The mice starting from a safety compartment were allowed to access to a drink providing compartment by way of passing the electronic current running compartment. If the passing number of mice through the electronic current running compartment increases, it is regarded that the impulsivity of mice gets stronger. The mice had been trained to endure the electronic current in order to drinking for 3 days and the supply of water had been stopped for 1 day. 90 mg/kg of NaCl was orally administrated into the mice causing to thirst at the testing day and then the test samples were administrated 1 hour after the administration. 30 mins after the administration of test samples, the passing number of mice was recorded by allowing the mice placing in a safety compartment to access to a drink providing compartment by way of passing the electronic current running compartment for 30 mins

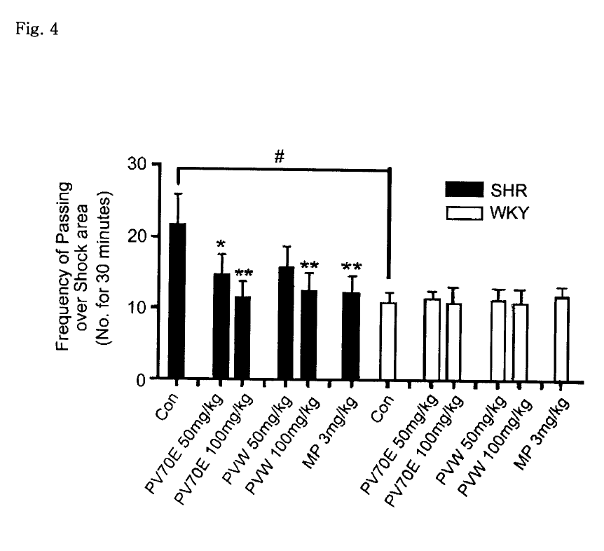

At the result, as can be seen in FIG. 4, the impulsivity behavior (=passing number through electric field) of SHR treated with the extract of Prunus vulgaris L prepared in Example 1 (PV70E and PVW) significantly reduced the increased impulsivity activity of the SHR mice in a dose dependent manner (significance; p<0.01), of which impulsivity behavior of the SHR mice was significantly increased comparing to normal mice. The impulsivity behavior of normal mice (WKY white mice) did not significantly changed and the impulsivity modulating capacity of SHR treated with the extract of Prunus vulgaris L prepared in Example 1 showed equivalent potency with that of SHR treated with methylphenidate used as a positive control.

It has been confirmed that the inventive extract of Prunus vulgaris L prepared in Example 1 (PV70E and PVW) showed potent inhibiting effect on the attention deficit, hyperactivity and impulsivity of the tested rats, whereas methylphenidate used as a positive control showed only inhibiting effect on the attention deficit and impulsivity behaviors of the tested mice while it did not showed any inhibiting effect on impulsivity behavior of the tested mice.

Accordingly, the inventive extract of Prunus vulgaris L prepared in Example 1 (PV70E and PVW) can be useful as a therapeutic agent or health care food for treating or preventing ADHD disease.

### EXPERIMENTAL EXAMPLE 2

### Toxicity Test

Methods

The acute toxicity tests on ICR mice (mean body weight 25±5 g) and Sprague-Dawley rats (235±10 g, Biogenomics Co., Ltd.) were performed by using the inventive extract of Prunus vulgaris L prepared in Example 1 (PV70E and PVW). Four group consisting of 3 mice or rats was administrated orally with 10 mg/kg, 100 mg/kg and 1000 mg/kg of test sample or solvents (0.2 ml, i.p.) respectively and observed for 2 weeks.

Results

There were no significant effects on mortality, clinical signs, body weight changes and gross findings in any group or either gender. These results suggested that the extract prepared in the present invention were potent and safe.

Hereinafter, the formulating methods and kinds of excipients will be described, but the present invention is not limited to them. The representative preparation examples were described as follows.

Preparation of powder

| | |
|---|---|
| PV70E | 50 mg |
| Lactose | 100 mg |
| Talc | 10 mg |

Powder preparation was prepared by mixing above components and filling sealed package.

Preparation of tablet

| | |
|---|---|
| PV70E | 50 mg |
| Corn Starch | 100 mg |
| Lactose | 100 mg |
| Magnesium Stearate | 2 mg |

Tablet preparation was prepared by mixing above components and entabletting.

Preparation of capsule

| | |
|---|---|
| PVW | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of injection

| | |
|---|---|
| PVW | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of liquid

| PVW | 0.1~80 g |
|---|---|
| Sugar | 5~10 g |
| Citric acid | 0.05~0.3% |
| Caramel | 0.005~0.02% |
| Vitamin C | 0.1~1% |
| Distilled water | 79~94% |
| CO₂ gas | 0.5~0.82% |

Liquid preparation was prepared by dissolving active component, filling all the components and sterilizing by conventional liquid preparation method.

Preparation of health food

| PV70E | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B6 | 0.5 | mg |
| Vitamin B12 | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-mentioned vitamin and mineral mixture may be varied in may ways. Such variations are not to be regarded as a departure from the spirit and scope of the present invention.

Preparation of health beverage

| | | |
|---|---|---|
| PVW | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

The inventive extract of Prunus vulgaris L showed potent inhibiting effect on the attention deficit, hyperactivity and impulsivity of the tested rats and no side effect or toxicity. Accordingly, the inventive extract of Prunus vulgaris L of the present invention can be useful as a therapeutic agent or health care food for treating or preventing ADHD disease.

1. A pharmaceutical composition comprising the extract of Prunus vulgaris, L as an active ingredient in an amount effective to treat or prevent ADHD disease, together with a pharmaceutically acceptable carrier.

2. The composition according to claim 1 wherein said extract is prepared by extracting a spike, herb, flower, or root, of Prunus vulgaris, L with polar solvent.

3. The composition according to claim 2 wherein said polar solvent is water, methanol, ethanol, butanol or the mixture thereof.

4. The composition according to claim 1 wherein said extract is prepared by the method comprising the step of; extracting the spike of Prunus vulgaris, L, with 1 to 100-fold volume of distilled water, lower alcohols or the mixtures thereof, at the temperature ranging from 30 to 110° C., for the period ranging from 1 hour to 5 hours, with the extraction method selected from reflux extraction or ultra-sonication extraction; filtering the residue to obtain the supernatant and concentrating and drying the supernatant to obtain dried extract powder of Prunus vulgaris L of claim 1.

5. The method for preparing the extract of Prunus vulgaris, L, comprising the step of; extracting the spike of Prunus vulgaris, L, with 1 to 100-fold volume of distilled water, lower alcohols such as methanol, ethanol, butanol and the like, or the mixtures thereof, at the temperature ranging from 30 to 110° C., for the period ranging from 1 hour to 5 hours, with the extraction method selected from reflux extraction or ultra-sonication extraction; filtering the residue to obtain the supernatant and concentrating and drying the supernatant to obtain dried extract powder of Prunus vulgaris L of claim 1.

6. A use of the extract ofPrunus vulgaris, L. in the manufacture of a medicament employed for treating or preventing ADHD disease in a mammal

7. A method of treating or preventing ADHD disease in a mammal wherein method comprises administering to said mammal an effective amount of the extract of Prunus vulgaris, L, together with a pharmaceutically acceptable carrier thereof.

8. A health care food comprising the extract of Prunus vulgaris, L, as an active ingredient in an amount effective to improve ADHD disease, together with a sitologically acceptable additive.

9. A health food or food additive comprising the extract of Prunus vulgaris, L to prevent and alleviate ADHD disease.

10. The health food or food additive according to claim 9 wherein said food or food additive is contained in candy, health beverage, gum, tea, vitamin complex, or dietary food.
